(19) United States
(12) Patent Application Publication
(10) Pub. No.: US 2006/0247522 A1
(43) Pub. Date: Nov. 2, 2006

(54) Magnetic navigation systems with dynamic mechanically manipulatable catheters

(75) Inventor: David L. McGee, Sunnyvale, CA (US)

Correspondence Address: Vista IP Law Group LLP, 2040 MAIN STREET, 9TH FLOOR, IRVINE, CA, 92614, US

(73) Assignee: Boston Scientific Scimed, Inc., Maple Grove, MN (US)

(21) Appl. No.: 11/117,031

(22) Filed: Apr. 28, 2005

(51) Int. Cl. A61M 25/00

(52) U.S. Cl. 600/434

(57) ABSTRACT

Systems and methods are provided for magnetically and mechanically navigating catheters. The system comprises a catheter that includes an elongated flexible catheter body having a distal end configured to be mechanically actuated to assume a non-compliant curved geometry. The distal end can be mechanically actuated in one of any number of manners. The catheter further comprises a magnetically responsive element and an operative element carried by the distal catheter end. The system further comprises a magnetic navigation system configured for applying a magnetic force to the magnetic element to deflect the distal catheter end. A method of using the system may comprise introducing the catheter within an anatomical cavity, mechanically actuating the distal catheter end to assume the curved geometry within the anatomical cavity, placing the operative element adjacent a target tissue site within the anatomical cavity, and performing a medical procedure on the target tissue site with the operative element. The magnetic navigation system can be used during catheter navigation and/or to firmly place the operative element against the target tissue site.

### FIELD OF THE INVENTION

The present inventions generally relate to medical navigation systems and methods, and more particularly to systems and methods for magnetically navigating catheters within a patient's body.

### BACKGROUND OF THE INVENTION

A number of companies have developed, or are developing, systems designed to magnetically manipulate and steer catheters (and other medical devices) inside the human body. In particular, a strong magnetic field is applied to the distal end of a catheter, which carries one or more magnetic elements (either permanent or electromagnetic magnets, or magnetic material, such as ferrous material), so that the resulting magnetic force moves the distal end of the catheter. The magnitude and direction of the magnetic force is determined by several factors: (a) the strength of the magnetic field; (b) the orientation (direction and polarity) of the magnetic field; and (c) the characteristics of the magnetic element(s) in the catheter. By controlling the strength and orientation of the magnetic field (e.g., using a gimbaled sets of electromagnets), the catheter can be steered within the body, and/or made to apply contact force to the tissue within the body.

To date, catheters designed to work with magnetic navigation systems have had very soft, floppy distal ends to readily orient in response to the magnetic forces applied by the navigation systems. Essentially, the current magnetically navigatable catheters have been “magnet on a rope” designs; the underlying thinking being that the distal end of the catheter can be entirely manipulated by controlling the characteristics (magnitude/orientation) of the applied magnetic field. Unfortunately, the real world performance of these designs may be sub-optimal due to the inherent limitations in current magnetic navigation system designs. Specifically, the magnetic fields generated by such systems cannot strictly control the position of the catheter tip, but rather can only impart a force (in a selected direction) to that catheter tip. The actual position of the catheter tip will be determined by the relationship between the force applied to the tip and any contact between the catheter and the tissue.

The limitations of conventional magnetic navigations systems are magnified when attempting to navigate catheters within three-dimensional anatomical cavities (i.e., cavities that have profiles much greater than the profile of the catheter), such as heart chambers. Because the distal ends of such catheters are somewhat floppy making their geometry unpredictable, the contact force applied to the catheters by the walls of the anatomical cavities makes accurate placement of these catheters, and in particular, the operate element(s) carried by their distal ends, at targeted tissue sites difficult to accomplish. Besides having difficulty navigating-a catheter within three-dimensional anatomical cavities, magnetic navigatable designs also cannot control the orientation of the catheter tip, and thus, the accompanying operative element(s), independently of the direction of the magnetic field. Instead, the catheter tip will tend align with the direction of the magnetic field. In cases where the orientation of an operative element may not matter, this will not be a problem. In many cases, however, it is desirable to orient the operative element relative to tissue in a particular manner, e.g., when attempting to place a lengthwise portion of an ablation catheter against the tissue to create a linear ablation lesion. It may be difficult to orient the ablation catheter in this manner using a conventional magnetic navigation system. In addition, the magnitude and direction of the magnetic force used to deflect the catheter tip in the desired direction must be constantly modified when attempting to locate the catheter tip at the desired location of the anatomical cavity.

Accordingly, there remains a need to be able to more efficiently and accurately use magnetic catheter navigation system to more accurately and efficiently navigate catheters within anatomical cavities.

### SUMMARY OF THE INVENTION

In accordance with a first aspect of the present inventions, a magnetic/mechanical catheter navigation system is provided. The system comprises a catheter that includes an elongated flexible catheter body having a distal end configured to be mechanically actuated to assume a non-compliant curved geometry. The distal end can be mechanically actuated in one of any number of manners. For example, the system can comprise a steering mechanism operable to actuate the distal catheter end to assume the curved geometry, or the system can comprise a stylet pre-shaped in the curved geometry and removably insertable within the catheter body to actuate the distal catheter end to assume the curved geometry. The catheter further comprises a magnetically responsive element carried by the distal catheter end. The magnetically responsive element can be any element that moves in response to a magnetic field, e.g., a permanent magnetic material, ferrous material, or electromagnet. The catheter further comprises an operative element (e.g., a tissue ablative element and/or diagnostic element) carried by the distal catheter end. The system further comprises a magnetic navigation system configured for applying a magnetic force to the magnetic element to deflect the distal catheter end.

In accordance with a second aspect of the present inventions, a method of performing a medical procedure in an anatomical cavity of a patient, such as a heart chamber, using the system described above is provided. The method comprises introducing the catheter within an anatomical cavity. The magnetic navigation system can optionally be operated to navigate the catheter into the anatomical cavity. The method further comprises mechanically actuating the distal catheter end to assume the curved geometry within the anatomical cavity, and placing the operative element adjacent a target tissue site within the anatomical cavity. The magnetic navigation system can optionally be operated to firmly place the operative element in contact with the target tissue site. The method further comprises performing a medical procedure on the target tissue site with the operative element.

In accordance with a third aspect of the present invention, another magnetic/mechanical navigation catheter system is provided. The system comprises a catheter that includes an elongated flexible catheter body having a distal end, and a magnetically responsive element and an operative element carried by the distal catheter end. The magnetically responsive element and operative element can have the same structure and function as those previously described. The system further comprises a mechanical steering mechanism configured for mechanically deflecting the catheter distal end, and a magnetic navigation system configured for magnetically deflecting the distal catheter end. The mechanical steering mechanism can either be a manual mechanism that is carried by the catheter, or an automatic mechanism contained within the magnetic navigation system.

In accordance with a fourth aspect of the present inventions, another method of performing a medical procedure in an anatomical cavity of a patient using previously described system is provided. This method is similar to the previous method, with the exception that it comprise operating the steering mechanism to deflect the catheter distal end within the anatomical cavity. The steering mechanism can optionally be operated to place the operative element in contact with the target tissue site.

In accordance with a fifth aspect of the present inventions, still another method of performing a medical procedure in a three-dimensional anatomical cavity of a patient, such as a heart chamber, is provided. The method comprises navigating a catheter through the vasculature of a patient, wherein a magnetic force is applied to deflect a distal end of the catheter during navigation. The method further comprises introducing the catheter within the anatomical cavity, and mechanically actuating the distal catheter end to assume a non-compliant curved geometry within the anatomical cavity. For example, a mechanical steering mechanism may be operated to actuate the distal catheter end, or a stylet may be inserted into the catheter to actuated the distal catheter end.

The method further comprises performing a medical procedure on a target tissue site within the anatomical cavity using the catheter. For example, the medical procedure may comprise creating an ablation lesion on the target tissue site and/or performing an electrophysiology mapping procedure on the target tissue site. In one method, the medical procedure may be performed on a linear region extending along the tissue target site without moving the catheter distal end. The catheter distal end may be placed into contact with the target tissue site during the performance of the medical procedure. For example, a magnetic force or internal mechanical force (e.g., using a mechanical steering mechanism or stylet) can be applied to the deflect the catheter distal end into firm contact with the target tissue site, or an internal mechanical force can be applied to the deflect the catheter.

In accordance with a sixth aspect of the present inventions, yet another method of performing a medical procedure in a three-dimensional anatomical cavity of a patient is provided. This method is similar to the previously described method, with the exception that instead of, or in addition to, applying a magnetic force to deflect the distal catheter end during navigation, the magnetic force is applied to deflect the distal catheter end into firm contact with the target tissue site.

Thus, it can be appreciated that the inventive system and method is capable of deflecting the distal end of the catheter using both a magnetic and a mechanical force. Although the present invention should not be so limited, the addition of mechanical navigation to conventional magnetic navigation system allows the catheter to be more efficiently and predictably navigated within an anatomical cavity, such as a heart chamber, and allows the operative elements to be more firmly placed in contact with a target tissue site within the anatomical cavity.

Other features of the present invention will become apparent from consideration of the following description taken in conjunction with the accompanying drawings.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of one preferred embodiment of a magnetic/mechanical catheter navigation system constructed in accordance with the present inventions;

FIG. 2 is a cross-sectional view of the ablation/mapping catheter, taken along the line 2-2 of FIG. 1;

FIG. 3 is a cross-sectional view of the ablation/mapping catheter of FIG. 2, taken along the line 3-3 of FIG. 1;

FIG. 4 is a cross-sectional view of the ablation/mapping catheter of FIG. 2, taken along the line 4-4 of FIG. 1;

FIG. 5 is a partially cutaway view of the distal end of the ablation/mapping catheter of FIG. 2, particularly showing one means for mechanically actuating the catheter;

FIG. 6 is a side view of the distal end of the ablation/mapping catheter of FIG. 2;

FIG. 7 is a side view of an alternative stylet that can be used to mechanically actuate the ablation/mapping catheter of FIG. 2; and

FIGS. 8A-8F are plan views of a method of using the magnetic/mechanical catheter navigation system of FIG. 1 to create a lesion within the right ventricle of a heart.

### DETAILED DESCRIPTION OF THE PREFERRED EMBODIMENTS

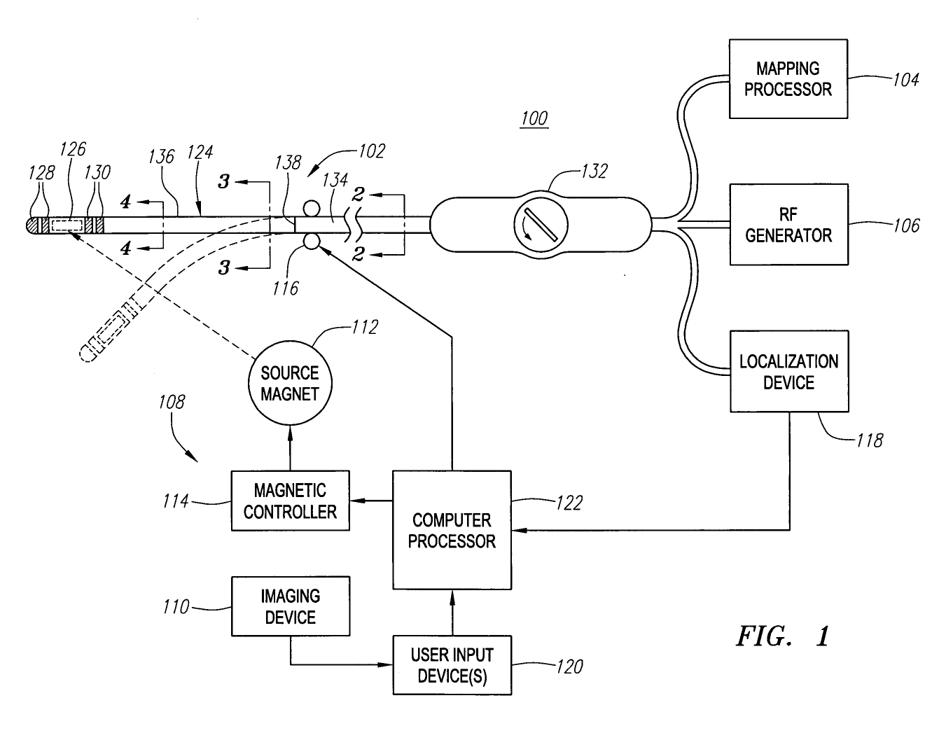

Referring to FIG. 1, a magnetic/mechanical catheter navigation system 100 constructed in accordance with the present inventions is shown. The system 100 generally comprises (a) an ablation/mapping catheter 102 configured to be introduced through the vasculature of the patient, and into a three-dimensional anatomical cavity, and in particular, a chamber of the heart, where it can be used to ablate and map heart tissue; (b) an electrophysiology mapping processor 104 used to electrophysiologically map heart tissue with the catheter 102 in order to identify arrhythmia causing substrates; (c) a source of ablation energy, and in particular, a radio frequency (RF) generator 106, for delivering ablation energy to the catheter 102 in order to ablate the identified substrates; and (d) a magnetic navigation system 108 for magnetically guiding the catheter 102 through the patient's vasculature and within the patient's heart. The system 100 may optionally comprise an introducer (not shown) for facilitating guidance of the catheter 102 within the patient's vasculature, in which case, the magnetic navigation system 108 would merely be used to manipulate the catheter 102 within the heart.

The mapping processor 104 is configured to detect, process, and record electrical signals within the heart. Based on these electrical signals, a physician can identify the specific target tissue sites within the heart to be ablated, and to ensure that the arrhythmia causing substrates within the heart have been destroyed by the ablative treatment. Such mapping techniques are well known in the art, and thus for purposes of brevity, will not be described in further detail. The RF generator 106 is configured to deliver ablation energy to the ablation/mapping catheter 102 in a controlled manner in order to ablate the target tissue sites identified by the mapping processor. Alternatively, other types of ablative sources besides the RF generator 106 can be used, e.g., a microwave generator, an ultrasound generator, a cryoablation generator, and a laser or other optical generator. Ablation of tissue within the heart is well known in the art, and thus for purposes of brevity, the RF generator 106 will not be described in further detail. Further details regarding RF generators are provided in U.S. Pat. No. 5,383,874, which is expressly incorporated herein by reference. It should be noted that although the mapping processor 104 and RF generator 106 are shown as discrete components, they can alternatively be incorporated into a single integrated device.

The magnetic navigation system 108 may be any conventional system that is capable of magnetically deflecting the distal end of the catheter 102. For example, as illustrated in FIG. 1, the magnetic navigation system 108 includes (a) an imaging device 110, such as a bi-planar fluoroscopic system; (b) a source magnet 112 for producing a magnetic field that deflects the distal end of the catheter 102; (c) a magnetic controller 114 for controlling the magnitude and direction of the magnetic field applied by the source magnet 112; (d) an advancing device 116 for longitudinally advancing and retracting the catheter 102; (e) a localization device 118 for registering the location of the catheter 102 within a three-dimensional coordinate system; (f) a user interface device or devices 120, such as keyboard, mouse, joystick, and display; and (g) a computer processor 122 for 1) obtaining the catheter location information from the localization device 118; 2) generating a graphical image of the catheter 102; 3) correlating the graphical catheter image 102 with a preoperative or graphical image of the anatomical cavity; and 4) controlling the magnetic controller 114 and advancing device 116 to deflect and advance the catheter distal end in accordance with the user input devices 120. Further details on one embodiment of the magnetic navigation system 108 are disclosed in U.S. Pat. No. 6,298,257, which is expressly incorporated herein by reference which can be correlated with the fluoroscopic image(s), a preoperative image, or a graphically generated image;

The ablation/mapping catheter 102 comprises an integrated flexible catheter body 124, a magnetically responsive element 126, a plurality of distally mounted operative elements, and in particular, a tissue ablative element 128 and a mapping element 130, and a proximally mounted handle 132. The catheter body 124 comprises a proximal member 134 and a distal member 136 that are preferably either bonded together at an interface 138 with an overlapping thermal bond or adhesively bonded together end to end over a sleeve in what is referred to as a “butt bond.” Alternatively, the integrated catheter body 124 may not have separate proximal and distal members 134,136 that are subsequently integrated together, but instead, may have an unibody design.

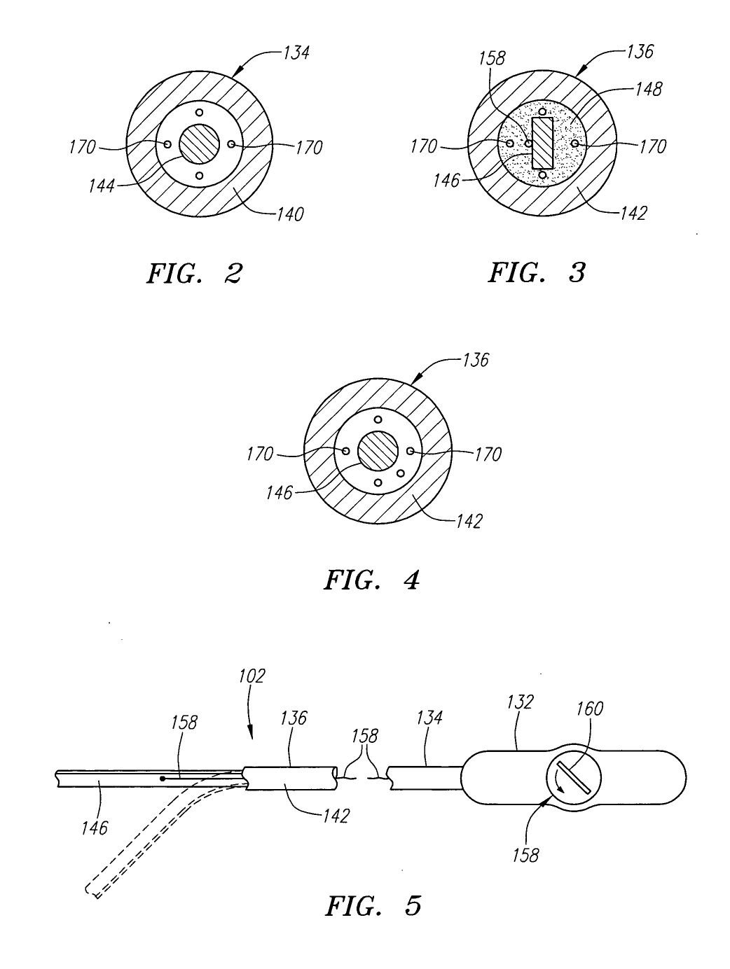

The catheter body 124 is preferably about 5 French to 9 French in diameter, with the proximal member 134 being relatively long (e.g., 80 cm to 100 cm), and the distal member 136 relatively short (e.g., 3 cm to 12 cm). As best illustrated in FIG. 2, the proximal member 134 comprises a tubular body 140 that is preferably formed from a biocompatible thermoplastic material, such as a Pebax® material (polyether block amide) and stainless steel braid composite, which has good torque transmission properties. In some implementations, an elongate guide coil (not shown) may also be provided within the proximal member 134. As best illustrated in FIGS. 3 and 4, the distal member 136 comprises a tubular body 142 that is preferably formed from a softer, more flexible biocompatible thermoplastic material such as unbraided Pebax® material, polyethylene, or polyurethane. The distal member 136 preferably includes a radio-opaque compound, such as barium, so that the catheter body 124 can be observed using fluoroscopic or ultrasound imaging, or the like. Alternatively, radio-opaque markers (not shown) can be placed along the distal member 136.

The catheter body 124 has a resilient shape that facilitates the functionality of the ablation/mapping catheter 102. In particular, and as is standard with most catheters, the proximal member 134 has an unconstrained straight or linear geometry to facilitate the pushability of the ablation/mapping catheter 102 through patient's vasculature, as well as to resist kinking. To this end, the proximal member 134 further comprises a resilient, straight center support 144 positioned inside of and passing through the length of the proximal tubular body 140. In the illustrated embodiment, the proximal center support 144 is a circular element formed from resilient inert wire, such as nickel titanium (commercially available under the trade name nitinol) or 17-7 stainless steel wire. Resilient injection molded plastic can also be used. The diameter of the proximal center support 144 is preferably between about 0.35 mm to 0.80 mm.

The distal member 136 is configured to be alternately placed between a linear geometry (shown in FIG. 1) and a curved geometry (shown in phantom in FIG. 1). The shape of the distal member 136 is achieved through the use of a center support 146 that is positioned inside of and passes through the length of the distal tubular body 142, as illustrated in FIG. 5. In the illustrated embodiment, the distal center support 146 is similar to the proximal center support 144 in composition and dimension. To improve the torqueability of the distal member 136, which is important to the predictable and controlled movement of the distal member 136, the distal center support 146 is preferably affixed within the distal portion of the proximal member 134 (such as by soldering the proximal end of the distal center support 146 to the distal end of the proximal center support 144), so that the torsional force applied to the proximal member 134 is transmitted to the distal member 136 without significant loss. Alternatively, the center supports 144, 146 can be formed of a unibody structure. To further improve the torqueability of the distal member 136, the proximal end of the center support 146 can be flattened into a rectangular cross-sectional geometry, as illustrated in FIG. 3. In addition, a filler material, such as epoxy 148, can be injected into the proximal end of the distal tubular body 142 in order to integrate all of the internal components of the distal member 136 together to further improve the torqueability at the junction between the proximal and distal members 134, 136.

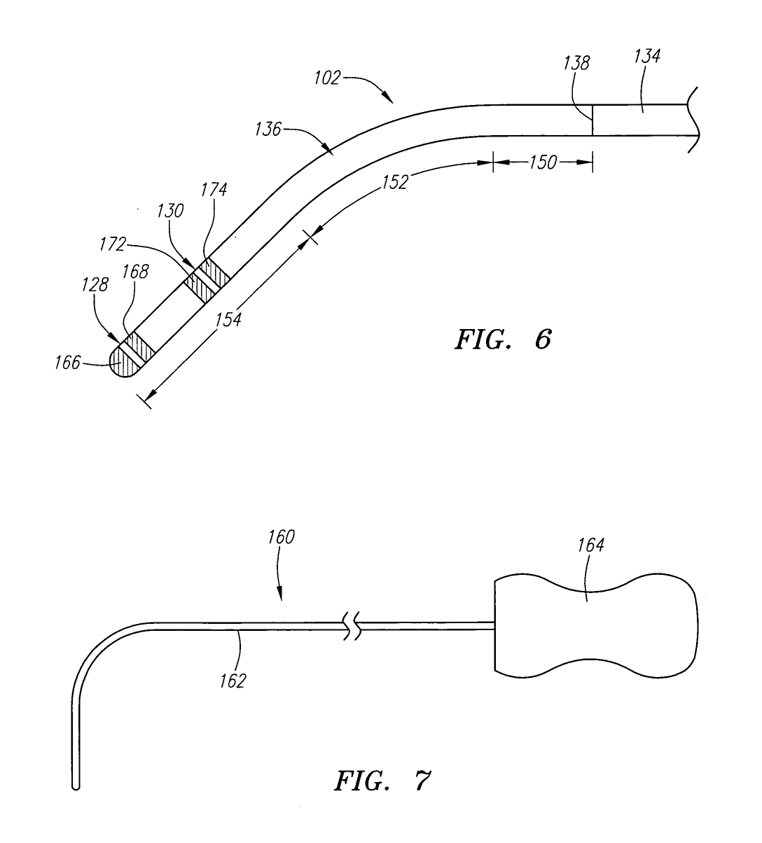

As best shown in FIG. 6, the distal member 136 has three geometrically distinct sections: (1) a shaft transition section 150 that distally extends from the proximal member; (2) a proximal section 152 that distally extends from the shaft transition section 150; and (3) a distal section 154 that distally extends from the proximal section 152.

The shaft transition section 150 is pre-shaped into a straight geometry. In the illustrated embodiment, the proximal member 134 and transition section 150 of the distal member 136 are collinear (i.e., the proximal member 134 and transition section 150 are not angled relative to each other). In this manner, bending forces that would otherwise be applied at the interface 138 between the proximal and distal members 138, 140 are minimized, thereby allowing more axial force to be applied to the ablation/mapping catheter 102 without collapsing the distal member 136 onto the proximal member 134 when proximal resistance is applied to the distal member 136.

The proximal section 152 is configured to be mechanically actuated from a straight geometry to form a simple curve (i.e., a curve that lies in a single plane) using the steering mechanism 156. In particular, as illustrated in FIG. 5, the catheter 102 comprises a steering mechanism 156 that is incorporated into the handle 132, and a steering wire 158 (shown also in FIG. 3) with its proximal end attached to the steering mechanism 156 and its distal end connected to the center support 146 at the interface between the proximal and intermediate sections 152, 154 of the distal member 136. The steering wire 158 is attached to the side of the center support 146 that faces the direction in which the proximal section 152 of the distal member 136 is configured to curve or bend (as shown in phantom).

The steering mechanism 156 comprises a rotatable steering lever 160, which when rotated in one direction, tensions the steering wire 158, thereby flexing the center support 146, and thus the proximal section 152 of the distal member 136, into the desired curve (shown in phantom). In contrast, rotation of the steering lever 160 in the opposite direction provides slack in the steering wire 158, thereby allowing the resiliency of the center support 146 to flex the proximal section 152 of the distal member 136 back into a straight geometry. Alternatively, the steering lever may be of the sliding type, wherein rearward movement of the steering lever flexes the center support 146, and thus the proximal section 152 of the distal member 136, into the desired curve, and forward movement of the steering lever allows the resiliency of the center support 146 to flex the proximal section 152 of the distal member 136 back into the straight geometry. Manually activated steering mechanisms for bending the distal ends of the catheters are well known in the prior art, and thus need not be described in further detail. Optionally, the steering mechanism can be automated, in which case, it can be incorporated into the magnetic navigation system 108 and controlled by the processor 122.

Although the steering mechanism 156 is described as unilaterally bending the proximal section 152 of the distal member 136 into the curved geometry, the steering mechanism 156 could be modified to bilaterally bending the proximal section 152 into two opposite curved geometry, e.g., by mounting another steering wire (not shown) to the side of the center support 146 opposite the first steering wire 158. In this case, rotation of the steering lever 160 in one direction tensions the first steering wire, thereby flexing the center support 146, and thus the proximal section 152 of the distal member 136, into a first desired curve in one direction, and rotation of the steering lever 160 in the opposite direction tensions the second steering wire, thereby flexing the center support 146, and thus the proximal section 152 of the distal member 136 into a second desired curve in the opposite direction. The opposite curves can either have the same geometry or may be different. Additional steering wires can be added to bend the proximal section 152 of the distal member 136 out-of-plane with the other curves.

It can be appreciated that the steering mechanism 156 provides internal navigational control over the distal member 136 of the catheter 102 in addition to the external control provided by the magnetic navigation system 108. As will be described in further detail below, this allows the catheter 102 to be more easily navigated within anatomical cavities. In addition, the steering mechanism 156 provides a more efficient means of properly placing the distal section 154 of the distal member 136, and thus, the ablative/mapping elements 128, 130, into firm contact with a target tissue site, as will be described in further detail below. Significantly, the steering mechanism 156 allows the distal member 136 of the catheter 102 to be placed into a known and repeatable curved geometry, so that a particular anatomical cavity can be more easily navigated by the catheter 102, and a tissue target site that is known to exist in a particular region of an anatomical cavity can be more efficiently and accurately mapped/ablated by the catheter 102. In addition, the combination of the center support 146 and tensioned steering wire 158 advantageously renders the curved distal member 136 non-compliant in that the distal member 136 will not easily bend from its known curved geometry when placed in firm contact with tissue. In this manner, the placement of ablative/mapping elements 128, 130 at a desired target tissue site can be more predictably controlled.

The use of a steering mechanism is not the only manner in which the distal member 136 of the catheter 102 can be placed into a non-compliant and predictable curved geometry. For example, as illustrated in FIG. 7, a stylet 160 can be used to selectively place the distal member 136 of the catheter 102 into the curved geometry. In particular, the stylet 160 comprises a shaft 162 have a pre-curved distal end and a handle 164 used to selectively insert the stylet 160 into a lumen (not shown) extending through the catheter body 124 to place the distal member 136 into its curved geometry, and removed from the lumen to place the distal member 136 into a floppy or straight geometry. Optionally, additional stylets 160 with differently curved distal ends can be provided, so that distal member 136 of the catheter 102 can be made to assume different curved geometries as desired.

The distal section 154 serves to carry the magnetically responsive element 126, as well as the ablative/mapping elements 128, 130, and is pre-shaped into a straight geometry, so that the ablative/mapping elements 128, 130 can be applied to the target tissue site in a linear fashion (i.e., a substantial length of the distal section 154 can be placed flush with tissue so that the lengths of the ablative/mapping elements 128, 130 can be placed against the tissue). Ultimately, the contour of the target tissue site will dictate the pre-shaped geometry of the distal section 154. For example, if the target tissue site exhibits an inwardly curved geometry (convex), the distal section 154 may have a pre-shaped geometry that curves in the same direction as the proximal section 152.

The magnetically responsive element 126 can take the form of an element that moves in response to a magnetic field. For example, the magnetically responsive element 126 can comprise a permanent magnetic material, such as neodymium-iron-boron, or can comprise a ferrous material, such as cold rolled steel or iron-cobalt alloy. The magnetically responsive element 126 can also take the form of an electromagnet connected to wires (not shown) that are passed in conventional fashion through a lumen (not shown) extending through the catheter body 124, where they are electrically coupled either directly to a connector (not shown) received in a port on the handle 132 or indirectly to the connector via a PC board (not shown) in the handle 132.

In the embodiment illustrated in FIG. 6, the ablative element 128 takes the form of a linear electrode assembly that includes a cap electrode 166 mounted to the distal tip of the distal member 136 and a ring electrode 168 mounted on the distal section 154 of the distal member 136 just proximal to the cap electrode 166. Notably, the split nature of the ablative element 128 provides selective monopolar and bipolar functionality to the catheter 102. That is, one or both of the tip/ring electrodes 166, 168 can be configured as one pole of a monopolar arrangement, so that ablation energy emitted by one or both of the electrodes 166, 168 is returned through an indifferent patch electrode (not shown) externally attached to the skin of the patient; or the tip/ring electrodes 166, 168 can be configured as two poles of a bipolar arrangement, in which energy emitted by one of the tip/ring electrodes 166, 168 is returned to the other electrode. In addition to serving as a selective unipolar/bipolar means of ablation, the tip/ring electrodes 166, 168 may also serve as a closely spaced high resolution pair of mapping electrodes. The combined length of the ablation electrodes 166, 168 is preferably about 6 mm to about 10 mm in length. In one embodiment, each ablation electrode is about 4 mm in length with 0.5 mm to 3.0 mm spacing, which will result in the creation of continuous lesion patterns in tissue when coagulation energy is applied simultaneously to the electrodes 166, 168.

The ablation electrodes 166, 168 may take the form of solid rings of conductive material, like platinum, or can comprise a conductive material, like platinum-iridium or gold, coated upon the device using conventional coating techniques or an ion beam assisted deposition (IBAD) process. For better adherence, an undercoating of nickel or titanium can be applied. Any combination of the electrodes can also be in the form of helical ribbons or formed with a conductive ink compound that is pad printed onto a nonconductive tubular body. A preferred conductive ink compound is a silver-based flexible adhesive conductive ink (polyurethane binder), however other metal-based adhesive conductive inks such as platinum-based, gold-based, copper-based, etc., may also be used to form electrodes. Such inks are more flexible than epoxy-based inks.

The ablation electrodes 166, 168 can alternatively comprise a porous material coating, which transmits coagulation energy through an electrified ionic medium. For example, as disclosed in U.S. Pat. No. 5,991,650, ablation electrodes may be coated with regenerated cellulose, hydrogel or plastic having electrically conductive components. With respect to regenerated cellulose, the coating acts as a mechanical barrier between the surgical device components, such as electrodes, preventing ingress of blood cells, infectious agents, such as viruses and bacteria, and large biological molecules such as proteins, while providing electrical contact to the human body. The regenerated cellulose coating also acts as a biocompatible barrier between the device components and the human body, whereby the components can now be made from materials that are somewhat toxic (such as silver or copper).

The ablation electrodes 166, 168 are electrically coupled to individual wires 170 (shown in FIGS. 2-4) to conduct ablation energy to them. The wires 170 are passed in conventional fashion through a lumen extending through the associated catheter body, where they are electrically coupled either directly to a connector (not shown) that is received in a port on the handle 132 or indirectly to the connector via a PC board (not shown) in the handle 132. The connector plugs into the RF generator 106 (shown in FIG. 1). Although ablation electrodes 166,168 have been described as the operative elements that create the lesion, other operative elements, such as elements for chemical ablation, laser arrays, ultrasonic transducers, microwave electrodes, and ohmically heated hot wires, and such devices may be substituted for the electrodes 166, 168.

The ablation/mapping catheter 102 further comprises temperature sensors (not shown), such as thermocouples or thermistors, which may be located on, under, abutting the longitudinal end edges of, or in between, the electrodes 166, 168. In some embodiments, a reference thermocouple (not shown) may also be provided. For temperature control purposes, signals from the temperature sensors are transmitted to the RF generator 106 by way of wires (not shown) that are also connected to the aforementioned PC board in the handle 132. Suitable temperature sensors and controllers, which control power to electrodes based on a sensed temperature, are disclosed in U.S. Pat. Nos. 5,456,682, 5,582,609 and 5,755,715.

In the embodiment illustrated in FIG. 6, the mapping element 116 takes the form of a pair of ring electrodes 172, 174 that are mounted on the distal section 154 of the distal member 136. Optionally, additional pairs of ring electrodes may be located along the distal member 136. The mapping electrodes 172, 174 are composed of a solid, electrically conducting material, like platinum or gold, attached about the catheter body 124. Alternatively, the mapping electrodes 172, 174 can be formed by coating the exterior surface of the catheter body 124 with an electrically conducting material, like platinum or gold. The coating can be applied using sputtering, ion beam deposition, or equivalent techniques. The mapping electrodes 172, 174 can have suitable lengths, such as between 0.5 and 5 mm. In use, the mapping electrodes 172, 174 sense electrical events in myocardial tissue for the creation of electrograms, and are electrically coupled to the mapping processor 104 (shown in FIG. 1). A signal wire 152 (shown in FIGS. 2-4) is electrically coupled to each mapping electrode 172, 174. The wires 152 extend through the catheter body 124 into an external multiple pin connector (not shown) located on the handle 132, which electrically couples the mapping electrodes 172, 174 to the mapping processor 104.

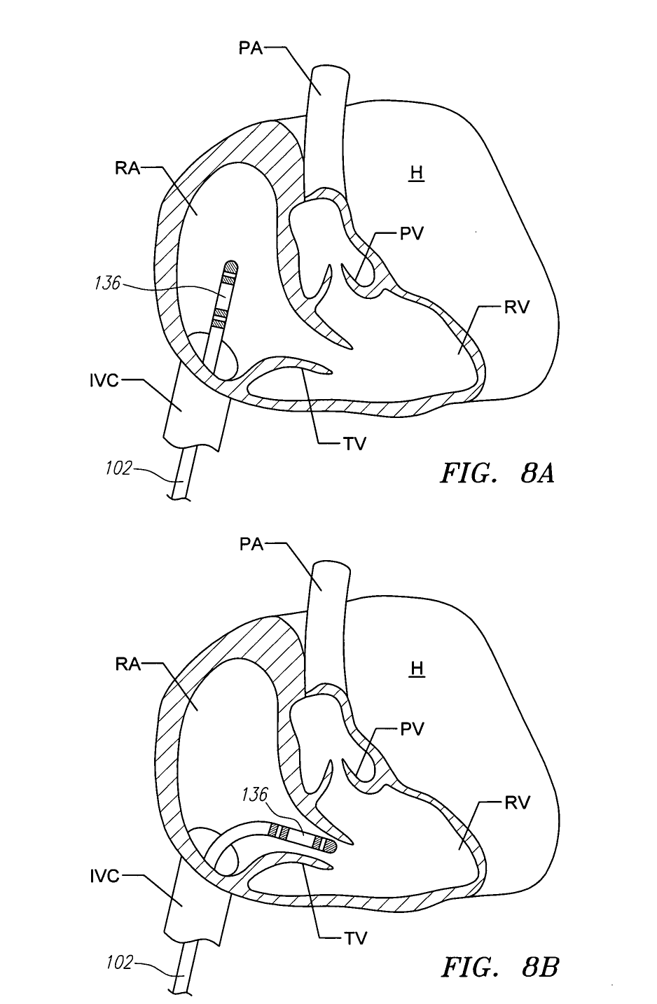

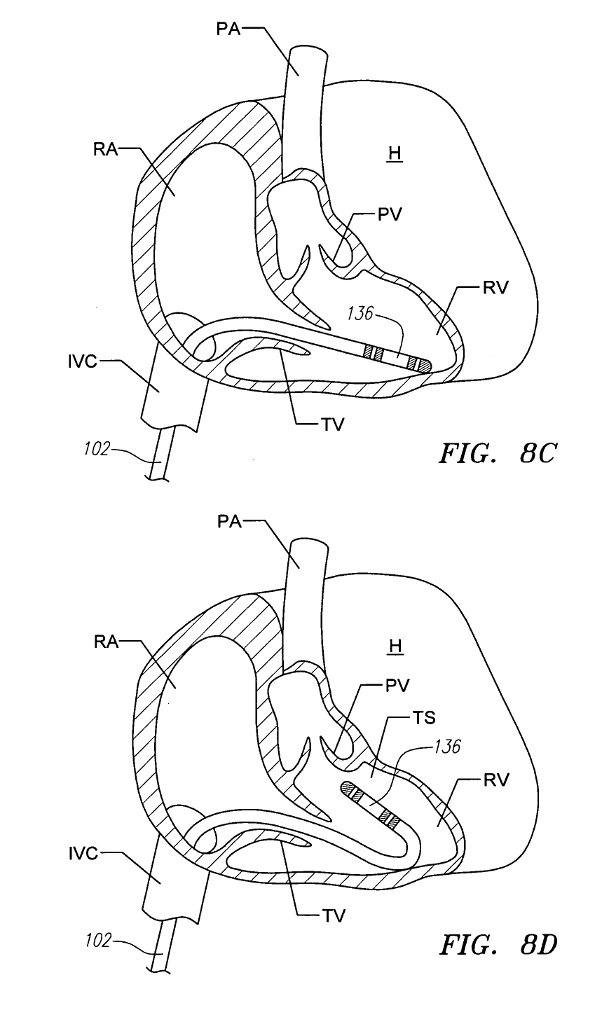

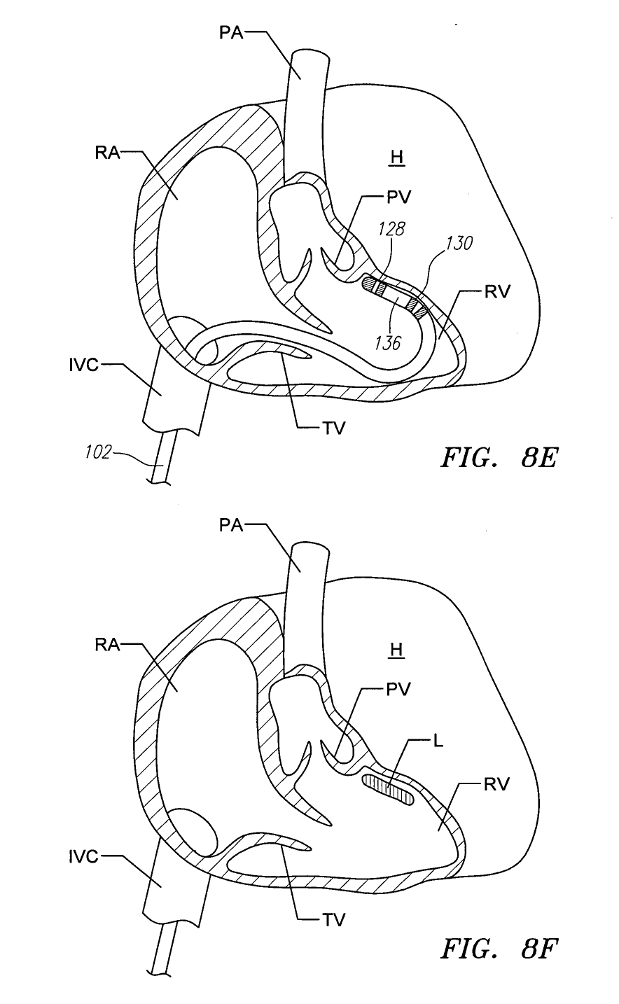

Having described the structure of the treatment system 100, its operation in identifying and destroying arrhythmia causing substrates within the right ventricle RV of a heart H, will now be described with reference to FIGS. 8A-8E. It should be noted that the views of the heart H and other interior regions of the body described herein are not intended to be anatomically accurate in every detail. The figures show anatomic details in diagrammatic form as necessary to show the features of the embodiment described herein.

First, the ablation/mapping catheter 102 is introduced up the inferior vena cava IVC until the distal member 136 resides within the right atrium RA of the heart H (FIG. 8A). Navigation of the catheter 102 into the heart H can be performed by operation of the magnetic navigation system 108 in a conventional manner. Once the distal catheter member 136 is properly located within the right atrium RA, the steering mechanism 156 is operated to deflect the distal member 136 towards and into the tricuspid valve TV leading to the right ventricle RV (FIG. 8B). The catheter 102 is then advanced so that the distal member 136 passes through the tricuspid valve TV and into the right ventricle RV (FIG. 8C). During this step, the steering mechanism 156 may be operated to straighten out the distal member 136, allowing the natural forces exerted by the tricuspid valve TV to guide the distal member 136 into the right ventricle RV Alternatively, rather than using the magnetic navigation system 108, a conventional guide sheath (not shown) can be used to introduce the catheter 102 into the right ventricle RV of the heart H.

Once the distal member 136 of the catheter 102 is properly placed in the right ventricle RV, the steering mechanism 156 is operated in order to deflect the distal catheter member 136 towards the pulmonary valve PV of the pulmonary artery PA (nearly 180 degrees from where it was directed prior to operation of the steering mechanism 156) where the target tissue site TS is located (FIG. 8D). If the steering mechanism 156 is only capable of unilateral deflection of the distal catheter member 136, the catheter may need to be rotated around its axis somewhat, so that the distal catheter member 136 deflects in the proper direction. If the steering mechanism 156 is capable of multi-lateral deflection of the distal catheter member 136, no such rotation is required. Minor adjustments to the position of the distal catheter member 136 can be made by operating the magnetic navigation system 108 in a conventional manner. The ablation/mapping elements 128, 130 are then firmly placed against the target tissue site TS (FIG. 8E). For example, the steering mechanism 156 can be operated to deflect the distal catheter member 136 towards the target tissue site TS and/or by magnetic navigation system 108 can be operated to apply a magnetic force in a direction towards the target tissue site TS, which causes the ablation/mapping elements 128, 130 to move towards and against the target tissue site TS.

It should be noted that if the stylet 160 illustrated in FIG. 7 is the preferred means of mechanically deflecting the distal catheter member 136, the stylet 160 can be inserted into the catheter 102 to deflect the distal catheter member 136 in the right atrium RA (as illustrated in FIG. 8B), then retracted or removed from the catheter 102 during introduction of the distal catheter member 136 into the right ventricle (as illustrated in FIG. 8C), and then inserted into the catheter 102 again to deflect the distal catheter member 136 in the right ventricle RV (as illustrated in FIG. 8D). A differently shaped stylet may alternatively be used to deflect the distal catheter member 136 within the right ventricle RV, and may be used to deflect the distal catheter member 136 into firm contact with the target tissue site TS (as illustrated in FIG. 8E).

In any event, once the ablation/mapping elements 128, 130 are firmly and stably in contact with the target tissue site TS, the mapping processor 104 (shown in FIG. 1) is operated in order to obtain and record ECG signals from the target tissue site TS, with the ablative element 128 serving as a mapping element to measure ECG signals in one region of the target tissue site TS, and the mapping element 116 serving to measure ECG signals in another region of the target tissue site TS. As described below, these ECG signals will be compared with the ECG signals obtained subsequent to an ablation procedure in order to determine if the resultant lesion has successfully destroyed the arrhythmia causing substrates in the right ventricle RV of the heart H.

Once the pre-ablation ECG signals have been obtained and recorded, the RF generator 106 (shown in FIG. 1) is operated in order to convey RF energy to the ablative element 128 (either in the monopolar or bipolar mode), thereby creating a linear lesion L (FIG. 8F). After the lesion L has been created, the mapping processor 104 is again operated to obtain and record ECG signals from the target tissue site TS. These post-ablation ECG signals are compared to the pre-ablation ECG signals to determine whether the arrhythmia causing substrates at the target tissue site TS have been destroyed. Once proper ablation has been confirmed, additional tissue target sites can be mapped and ablated, e.g., by moving the ablation/mapping elements 128, 130 away from the original target tissue site TS (via operation of the steering mechanism 156 or magnetic navigation system 108) and manipulating the catheter (e.g., by rotation) to place the ablation/mapping elements 128, 130 at another target tissue site. The steps illustrated in FIGS. 8D-8F can then be repeated.

Although particular embodiments of the present invention have been shown and described, it will be understood that it is not intended to limit the present invention to the preferred embodiments, and it will be obvious to those skilled in the art that various changes and modifications may be made without departing from the spirit and scope of the present invention. Thus, the present inventions are intended to cover alternatives, modifications, and equivalents, which may be included within the spirit and scope of the present invention as defined by the claims.

What is claimed is:

1. A magnetic/mechanical catheter navigation system, comprising:
a catheter, including:
an elongated flexible catheter body having a distal end, at least portion of which is configured to be mechanically actuated to assume a non-compliant curved geometry;
a magnetically responsive element carried by the distal catheter end; and
an operative element carried by the distal catheter end;
a magnetic navigation system configured for applying a magnetic force to the magnetic element to deflect the distal catheter end.

2. The catheter of claim 1, wherein the operative element comprises a tissue ablative element.

3. The catheter of claim 1, wherein the operative element comprises a diagnostic element.

4. The catheter of claim 1, wherein the operative element longitudinally extends along at least portion of the distal catheter end.

5. The catheter of claim 1, further comprising a steering mechanism operable to actuate the distal catheter end to assume the curved geometry.

6. The catheter of claim 1, further comprising a stylet pre-shaped in the curved geometry and removably insertable within the catheter body to actuate the distal catheter end to assume the curved geometry.

7. A method of performing a medical procedure in an anatomical cavity of a patient using the system of claim 1, comprising:
introducing the catheter within an anatomical cavity;
mechanically actuating at least a portion of the distal catheter end to assume the curved geometry within the anatomical cavity;
placing the operative element adjacent a target tissue site within the anatomical cavity; and
performing a medical procedure on the target tissue site with the operative element.

8. The method of claim 7, further comprising operating the magnetic navigation system to navigate the catheter into the anatomical cavity.

9. The method of claim 7, further comprising operating the magnetic navigation system to firmly place the operative element in contact with the target tissue site.

10. The method of claim 7, wherein the anatomical cavity is a heart chamber.

11. A magnetic/mechanical catheter navigation system, comprising:
a catheter, including:
an elongated flexible catheter body having a distal end;
a magnetically responsive element carried by the distal catheter end; and
an operative element carried by at least portion of the distal catheter end;
a mechanical steering mechanism configured for mechanically deflecting the catheter distal end;
a magnetic navigation system configured for magnetically deflecting the distal catheter end.

12. The catheter of claim 11, wherein the operative element comprises a tissue ablative element.

13. The catheter of claim 11, wherein the at least one operative element comprises a diagnostic element.

14. The catheter of claim 11, wherein the operative element longitudinally extends along the distal catheter end.

15. The catheter of claim 11, wherein the mechanical steering mechanism is carried by the catheter and is configured to be manually operated.

16. The catheter of claim 11, wherein the mechanical steering mechanism is contained within the magnetic navigation system, the magnetic navigation system configured for automatically operating the steering mechanism.

17. A method of performing a medical procedure in an anatomical cavity of a patient using the system of claim 11, comprising:
introducing the catheter within an anatomical cavity;
operating the steering mechanism to deflect the catheter distal end within the anatomical cavity;
placing the operative element adjacent a target tissue site within the anatomical cavity; and
performing a medical procedure on the target tissue site with the operative element.

18. The method of claim 11, further comprising operating the magnetic navigation system to navigate the catheter into the anatomical cavity.

19. The method of claim 11, further comprising operating the magnetic navigation system to firmly place the operative element in contact with the target tissue site.

20. The method of claim 11, further comprising operating the steering mechanism again to firmly place the operative element in contact with the target tissue site.

21. The method of claim 11, wherein the anatomical cavity is a heart chamber.

22. A method of performing a medical procedure in a three-dimensional anatomical cavity of a patient using the system of claim 1, comprising:
introducing the catheter within anatomical cavity;
operating the magnetic navigation system to apply the magnetic force to the magnetic element to move the distal catheter end towards a target tissue site within the anatomical cavity;
internally actuating the distal catheter end from the straight geometry to the curved geometry, whereby the operative element moves adjacent the target tissue site; and
performing a medical procedure on the target tissue site with the operative element.

23. The method of claim 22, wherein the operative element is placed into contact with the target tissue site.

24. The method of claim 22, wherein the operative element is a linear ablation element, and the medical procedure comprises creating a linear ablation on the target tissue site with the ablation element.

25. The method of claim 22, wherein the anatomical cavity is a heart chamber.

26. A method of performing a medical procedure in a three-dimensional anatomical cavity of a patient, comprising:
navigating a catheter through the vasculature of a patient, wherein a magnetic force is applied to deflect a distal end of the catheter during navigation;
introducing the catheter within the anatomical cavity;
mechanically actuating at least a portion of the distal catheter end to assume a non-compliant curved geometry within the anatomical cavity; and
performing a medical procedure on a target tissue site within the anatomical cavity using the catheter.

27. The method of claim 26, wherein the medical procedure comprises creating an ablation lesion on the target tissue site.

28. The method of claim 26, wherein the medical procedure comprises performing an electrophysiology mapping procedure on the target tissue site.

29. The method of claim 26, wherein the medical procedure is performed on a linear region extending along the tissue target site without moving the catheter distal end.

30. The method of claim 26, wherein a mechanical steering mechanism is operated to actuate the distal catheter end.

31. The method of claim 26, wherein a stylet is inserted into the catheter to actuate the distal catheter end.

32. The method of claim 26, wherein the catheter distal end is placed into contact with the target tissue site.

33. The method of claim 32, wherein a magnetic force is applied to the deflect the catheter distal end into firm contact with the target tissue site.

34. The method of claim 32, wherein an internal mechanical force is applied to the deflect the catheter distal end into firm contact with the target tissue site.

35. The method of claim 26, wherein the anatomical cavity is a heart chamber.

36. A method of performing a medical procedure in a three-dimensional anatomical cavity of a patient, comprising:
introducing the catheter within the anatomical cavity;
mechanically actuating the distal catheter end to assume a non-compliant curved geometry within the anatomical cavity;
placing the distal catheter end into contact with a target tissue site within the anatomical cavity;
applying a magnetic force to deflect the distal catheter end into firm contact with the target tissue site; and
performing a medical procedure on the target tissue site with the catheter distal end.

37. The method of claim 36, wherein the medical procedure comprises creating an ablation lesion on the target tissue site.

38. The method of claim 36, wherein the medical procedure comprises performing an electrophysiology mapping procedure on the target tissue site.

39. The method of claim 36, wherein the medical procedure is performed on a linear region extending along the tissue target site without moving the catheter distal end.

40. The method of claim 36, wherein a mechanical steering mechanism is operated to actuate the distal catheter end.

41. The method of claim 36, wherein a stylet is inserted into the catheter to actuate the distal catheter end.

42. The method of claim 36, further comprising navigating a catheter through the vasculature of a patient, wherein a magnetic force is applied to deflect the catheter distal end during navigation.

43. The method of claim 36, wherein the anatomical cavity is a heart chamber.
